SEQUENCE LISTING (14 sequences)

SEQ ID NO: 1 - DNA, 1539 bp, Canis familiaris
atggcgttctcccagtatatctccttagccccagagctgctactggccactgccatcttctgtttagtgttctgggtgttgagaggcacaaggacccaggttcccaaaggtctgaagagtcctcccggaccctggggcttgcccttcattgggcacatgctgaccctggggaagaacccacacctatctctgacaaagctgagtcagcagtatggggacgtgctgcagatccgcattggctccacacccgtggtggtgctgagcggcctgaacaccatcaagcaggccctagtgaagcagggggatgacttcaaaggccggccagacctctacagcttcacacttatcactaatggcaagagcatgactttcaacccagactctggaccggtctgggctgcccgccggcgcctggcccaggatgccctgaagagtttctccatagcctcagaccccacatcagtatcctcttgctacttggaggagcacgtgagcaaagaggctaaccatctaatcagcaagttccagaagctgatggcagaggttggccacttcgaaccagtcaaccaggtggtggaatcggtggctaatgtcatcggagccatgtgttttgggaagaacttccccaggaagagcgaggagatgctcaacctcgtgaagagcagcaaggactttgtggagaatgtcacctcagggaatgctgtggacttctttccggtcctgcgctacctgcccaacccagccctcaagaggtttaagaacttcaatgataactttgtgctgtttctgcagaaaacagtccaggaacactatcaagacttcaacaagaacagtatccaggacatcacaggcgccctgttcaagcacagtgagaactacaaagacaacggtggtctcatccctcaggagaagattgtcaacattgtcaatgacatctttggagctggatttgaaacagtcacaacagccatcttctggagcattttgctacttgtgacagagcccaaggtgcagaggaagattcatgaggagctggacacggtgattggcagagatcggcagccacggctttctgacagaccccagctgccatatctggaggccttcatcctggagatctaccgatacacatcctttgtccccttcaccatcccccacagcacaacgagggacacctcactgaatggcttccacattcccaaggagtgctgcatcttcataaaccagtggcaggtcaaccatgatgagaagcagtggaaagacccctttgtgttccgcccagagcggtttcttaccaatgacaacacggccatcgacaagaccctgagtgagaaggtgatgctcttcggcttgggaaagcgccggtgcattggggagatcccggccaagtgggaagtcttcctcttcttagccatcctcctgcatcagctggagttcactgtgccaccgggcgtgaaggtggacctgacacccagctatgggctgaccatgaagcccagaacctgtgaacacgtccaggcctggccacgcttctccaag

SEQ ID NO: 2 - protein, 513 aa, Canis familiaris
MAFSQYISLAPELLLATAIFCLVFWVLRGTRTQVPKGLKSPPGPWGLPFIGHMLTLGKNPHLSLTKLSQQYGDVLQIRIGSTPVVVLSGLNTIKQALVKQGDDFKGRPDLYSFTLITNGKSMTFNPDSGPVWAARRRLAQDALKSFSIASDPTSVSSCYLEEHVSKEANHLISKFQKLMAEVGHFEPVNQVVESVANVIGAMCFGKNFPRKSEEMLNLVKSSKDFVENVTSGNAVDFFPVLRYLPNPALKRFKNFNDNFVLFLQKTVQEHYQDFNKNSIQDITGALFKHSENYKDNGGLIPQEKIVNIVNDIFGAGFETVTTAIFWSILLLVTEPKVQRKIHEELDTVIGRDRQPRLSDRPQLPYLEAFILEIYRYTSFVPFTIPHSTTRDTSLNGFHIPKECCIFINQWQVNHDEKQWKDPFVFRPERFLTNDNTAIDKTLSEKVMLFGLGKRRCIGEIPAKWEVFLFLAILLHQLEFTVPPGVKVDLTPSYGLTMKPRTCEHVQAWPRFSK

SEQ ID NO: 3 - DNA, 30 bp, Artificial
  Primer Sequence
atggcgttctcccagtatatctccttagcc

SEQ ID NO: 4 - DNA, 30 bp, Artificial
  Primer Sequence
tcacttggagaagcgtggccaggcctggac

SEQ ID NO: 5 - DNA, 22 bp, Artificial
  Primer Sequence
ctgcttcactagggcctgcttg

SEQ ID NO: 6 - DNA, 23 bp, Artificial
  Primer Sequence
gcaaagaggctaaccatctaatc

SEQ ID NO: 7 - DNA, 24 bp, Artificial
  Primer Sequence
gcaaaatgctccagaagatggctg

SEQ ID NO: 8 - DNA, 22 bp, Artificial
  Primer Sequence
ggccttcatcctggagatctac

SEQ ID NO: 9 - DNA, 2169 bp, Homo sapiens
atggccgagtatacgcggctgcacaacgccttggcgctaatccgcctccgaaacccgccggtcaacgcgatcagtacgactttactccgtgatataaaagaaggactacagaaagctggaagagaccatacaataaaagccattgtgatttgtggagcagagggcaaattttctgcaggtgctgatattcgtggcttcagtgctcctaggacatttggccttatactgggacatgtagtagatgaaatacagagaaatgagaagcccgtggtggcagcaatccaaggcatggctttcggagggggactagagctggccctgggctgtcactataggattgcccacgcagacgctcaagttggcttaccagaagttacacttggacttctccctggtgcaagaggaacccagcttctccccagactcactggagttcctgctgcacttgacttaattacctcaggaagacgtattttagcagatgaagcactcaagctgggcattctagataaagttgtaaactcagacccggttgaagaagcaatcagatttgctcagagagtttcagatcaacctctagaatcccgtagactctgcaacaagccaattcagagcttgcccaacatggacagcatttttagtgaggccctcttgaagatgcggaggcagcaccctgggtgtcttgcacaggaggcttgtgtccgtgcagtccaggctgctgtgcagtatccctatgaagtgggcatcaagaaggaggaggagctgtttctatatcttttgcaatcagggcaggctagagccctgcaatatgctttcttcgctgaaaggaaagcaaataagtggtcaactccctccggagcatcgtggaaaacagcatcagcgcggcctgtctcctcagttggtgttgttggcttgggaacaatgggccgaggcattgtcatttcttttgcaagggccaggattcctgtgattggtgtagactcggacaaaaaccagctagcaactgcaaacaagatgataacctctgtcttggaaaaagaagcctccaaaatgcaacagagcggccacccttggtcaggaccaaaacccaggttaacttcatctgtgaaggagcttggtggtgtagatttagtcattgaagcagtatttgaggaaatgagcctgaagaagcaggtctttgctgaactctcagctgtgtgcaaaccagaagcatttttgtgcactaatacttcagccctggatgttgatgagattgcttcttccactgatcgtcctcacttggtcattggcacccacttcttttcgccagctcatgtcatgaagttgttagaggttattcccagccaatactcttcccccactaccattgccactgttatgaacttatcaaaaaagattaaaaagattggagtcgttgtaggcaactgttttggatttgtggggaatcgaatgttgaatccttactacaatcaggcatatttcttgttagaagaaggcagcaaaccagaggaggtagatcaggtgctggaagagtttggttttaaaatgggaccttttagagtgtctgatcttgctgggttggatgtgggctggaaatctagaaaggggcaaggtcttactggacctacattgcttccaggaactcctgcccgaaaaaggggtaataggaggtactgcccaattcctgatgtgctctgtgaattaggacgatttggccagaagacaggtaagggttggtatcaatatgacaagccattgggtaggattcacaaacctgatccctggctttccacattcctatcacggtatagaaaaccccatcacattgaaccacgtaccattagccaggatgagatccttgaacgctgcttatattcacttatcaatgaagcattccgtatcttgggagaagggatagctgctagcccagagcacattgatgttgtctatttacatggatatggatgcgcaaggcacaagggcgggcccatgttctatgcttccacagttgggttgcccacagttctagagaaattgcagaaatattacaggcagaaccctgatattccccaactggagccaagtgactatctaaaaaaactggcttctcagggaaaccctcccctgaaagaatggcaaagcttggcaggctcccctagcagtaaattg

SEQ ID NO: 10 - protein, 515 aa, Homo sapiens
MALSQSVPFSATELLLASAIFCLVFWVLKGLRPRVPKGLKSPPEPWGWPLLGHVLTLGKNPHLALSRMSQRYGDVLQIRIGSTPVLVLSRLDTIRQALVRQGDDFKGRPDLYTSTLITDGQSLTFSTDSGPVWAARRRLAQNALNTFSIASDPASSSSCYLEEHVSKEAKALISRLQELMAGPGHFDPYNQVVVSVANVIGAMCFGQHFPESSDEMLSLVKNTHEFVETASSGNPLDFFPILRYLPNPALQRFKAFNQRFLWFLQKTVQEHYQDFDKNSVRDITGALFKHSKKGPRASGNLIPQEKIVNLVNDIFGAGFDTVTTAISWSLMYLVTKPEIQRKIQKELDTVIGRERRPRLSDRPQLPYLEAFILETFRHSSFLPFTIPHSTTRDTTLNGFYIPKKCCVFVNQWQVNHDPELWEDPSEFRPERFLTADGTAINKPLSEKMMLFGMGKRRCIGEVLAKWEIFLFLAILLQQLEFSVPPGVKVDLTPIYGLTMKHARCEHVQARRFSIN

SEQ ID NO: 11 - DNA, 1539 bp, Mus musculus
atggcgttctcccagtacatctccttagccccagagctgctactggccactgccatcttctgtttagtgttctggatggtcagagcctcaaggacccaggttcccaaaggcctgaagaatccacccggaccctggggcttgcccttcattgggcacatgctgactgtggggaagaacccacacctgtcactgacacggctgagtcagcagtatggggacgtgctgcagatccgcatcggctccactcctgtggtggtgctgagcggcctgaacaccatcaagcaggccctggtgaggcagggagatgacttcaagggccgaccagacctctacagcttcacacttatcactaacggcaagagcatgactttcaacccagactctggacccgtgtgggctgcccgccggcgcctggcccaggatgccctgaagagcttctccatagcctcggacccgacgtcagcatcctcttgctatttggaggagcacgtgagcaaggaggctaaccatctcgtcagcaagcttcagaaggcgatggcagaggttggccacttcgaaccagtcagccaggtggtggaatcggtggctaacgtcattggtgccatgtgctttgggaagaacttcccccggaagagcgaggagatgctgaacatcgtgaataacagcaaggactttgtggagaatgtcacctcagggaatgcagtggacttcttcccggtcctgcgctacctgcccaacccggccctcaagaggtttaagaccttcaatgataacttcgtgctgtttctgcagaaaactgtccaggagcactaccaagacttcaacaagaacagtatccaagacatcacaagtgccctgttcaagcacagcgagaactacaaagacaatggcggtctcatccccgaggagaagattgtcaacattgtcaatgacatctttggagctggctttgacacagtcaccacagccatcacctggagcattttgctacttgtgacatggcctaacgtgcagaggaagatccatgaggagctggacacggtggttggcagggatcggcaaccacggctttctgaccgtccccagctgccatatctagaggccttcatcctggagatctaccgatacacatcctttgtccccttcaccatcccccacagcacaacgagggacacctcactgaatggcttccacattcccaaggagcgctgtatctacataaaccagtggcaggtcaaccatgatgagaagcagtggaaagacccctttgtgttccgcccagagcggtttcttaccaataacaactcggccatcgacaagacccagagcgagaaggtgatgctcttcggcttgggaaagcgccggtgcattggggagatcccggccaagtgggaagtcttcctcttcttagccatcctgctgcagcatctggagtttagtgtgccaccgggtgtgaaggtggacctgacacccaactatgggttgaccatgaagcccgggacctgtgaacacgtccaggcatggccacgcttttccaag

SEQ ID NO: 12 - protein, 513 aa, Mus musculus
MAFSQYISLAPELLLATAIFCLVFWMVRASRTQVPKGLKNPPGPWGLPFIGHMLTVGKNPHLSLTRLSQQYGDVLQIRIGSTPVVVLSGLNTIKQALVRQGDDFKGRPDLYSFTLITNGKSMTFNPDSGPVWAARRRLAQDALKSFSIASDPTSASSCYLEEHVSKEANHLVSKLQKAMAEVGHFEPVSQVVESVANVIGAMCFGKNFPRKSEEMLNIVNNSKDFVENVTSGNAVDFFPVLRYLPNPALKRFKTFNDNFVLFLQKTVQEHYQDFNKNSIQDITSALFKHSENYKDNGGLIPEEKIVNIVNDIFGAGFDTVTTAITWSILLLVTWPNVQRKIHEELDTVVGRDRQPRLSDRPQLPYLEAFILEIYRYTSFVPFTIPHSTTRDTSLNGFHIPKERCIYINQWQVNHDEKQWKDPFVFRPERFLTNNNSAIDKTQSEKVMLFGLGKRRCIGEIPAKWEVFLFLAILLQHLEFSVPPGVKVDLTPNYGLTMKPGTCEHVQAWPRFSK

SEQ ID NO: 13 - DNA, 1539 bp, Rattus norvegicus
atggcgttctcccagtatatctccttagccccagagctgctactggccactgccatcttctgtttagtgttctgggtgttgagaggcacaaggacccaggttcccaaaggtctgaagagtcctcccggaccctggggcttgcccttcattgggcacatgctgaccctggggaagaacccacacctatctctgacaaagctgagtcagcagtatggggacgtgctgcagatccgcattggctccacacccgtggtggtgctgagcggcctgaacaccatcaagcaggccctagtgaagcagggggatgacttcaaaggccggccagacctctacagcttcacacttatcactaatggcaagagcatgactttcaacccagactctggaccggtctgggctgcccgccggcgcctggcccaggatgccctgaagagtttctccatagcctcagaccccacatcagtatcctcttgctacttggaggagcacgtgagcaaagaggctaaccatctaatcagcaagttccagaagctgatggcagaggttggccacttcgaaccagtcaaccaggtggtggaatcggtggctaatgtcatcggagccatgtgttttgggaagaacttccccaggaagagcgaggagatgctcaacctcgtgaagagcagcaaggactttgtggagaatgtcacctcagggaatgctgtggacttctttccggtcctgcgctacctgcccaacccagccctcaagaggtttaagaacttcaatgataactttgtgctgtctctgcagaaaacagtccaggaacactatcaagacttcaacaagaacagtatccaggacatcacaggcgccctgttcaagcacagtgagaactacaaagacaacggtggtctcatccctcaggagaagattgtcaacattgtcaatgacatctttggagctggatttgaaacagtcacaacagccatcttctggagcattttgctacttgtgacagagcccaaggtgcagaggaagattcatgaggagctggacacggtgattggcagagatcggcagccacggctttctgacagaccccagctgccatatctggaggccttcatcctggagatctaccgatacacatcctttgtccccttcaccatcccccacagcacaacgagggacacctcactgaatggcttccacattcccaaggagtgctgcatcttcataaaccagtggcaggtcaaccatgatgagaagcagtggaaagacccctttgtgttccgcccagagcggtttcttaccaatgacaacacggccatcgacaagaccctgagtgagaaggtgatgctcttcggcttgggaaagcgccggtgcattggggagatcccggccaagtgggaagtcttcctcttcttagccatcctcctgcatcagctggagttcactgtgccaccgggcgtgaaggtggacctgacacccagctatgggctgaccatgaagcccagaacctgtgaacacgtccaggcctggccacgcttctccaag

SEQ ID NO: 14 - protein, 513 aa, Rattus norvegicus
MAFSQYISLAPELLLATAIFCLVFWVLRGTRTQVPKGLKSPPGPWGLPFIGHMLTLGKNPHLSLTKLSQQYGDVLQIRIGSTPVVVLSGLNTIKQALVKQGDDFKGRPDLYSFTLITNGKSMTFNPDSGPVWAARRRLAQDALKSFSIASDPTSVSSCYLEEHVSKEANHLISKFQKLMAEVGHFEPVNQVVESVANVIGAMCFGKNFPRKSEEMLNLVKSSKDFVENVTSGNAVDFFPVLRYLPNPALKRFKNFNDNFVLSLQKTVQEHYQDFNKNSIQDITGALFKHSENYKDNGGLIPQEKIVNIVNDIFGAGFETVTTAIFWSILLLVTEPKVQRKIHEELDTVIGRDRQPRLSDRPQLPYLEAFILEIYRYTSFVPFTIPHSTTRDTSLNGFHIPKECCIFINQWQVNHDEKQWKDPFVFRPERFLTNDNTAIDKTLSEKVMLFGLGKRRCIGEIPAKWEVFLFLAILLHQLEFTVPPGVKVDLTPSYGLTMKPRTCEHVQAWPRFSK

(19) United States
(12) Patent Application Publication
(10) Pub. No.: US 2004/0158043 A1
(43) Pub. Date: Aug. 12, 2004

(54) Canine CYP1A2 sequences

(76) Inventor: Yizhong Gu, Wharton, NJ (US)

Correspondence Address: PHARMACIA & UPJOHN, 301 HENRIETTA ST, 0228-32-LAW, KALAMAZOO, MI, 49007, US

(21) Appl. No.: 10/744,150

(22) Filed: Dec. 22, 2003

Related U.S. Application Data
(60) Provisional application No. 60/437,602, filed on Dec. 31, 2002.

(51) Int. Cl. C12Q 1/68; C07H 21/04; C12N 5/06; C07K 14/52

(52) U.S. Cl. 530/351; 530/388.23; 435/6; 435/69.1; 435/350; 536/23.5

(57) ABSTRACT

The present invention provides a cDNA encoding a heretofore unknown polypeptide termed canine CYP1A2; the canine CYP1A2 polypeptide encoded by the gene; antibodies to the polypeptide; and methods of making and using all of the foregoing.

### CROSS REFERENCE TO RELATED APPLICATIONS

This application claims the benefit of the following provisional application: Application Serial No. 60/437,602 filed Dec. 31, 2002 under 35 U.S.C. 119(e)(1), which is incorporated herein by reference in its entirety.

### FIELD OF THE INVENTION

The present invention provides isolated polypeptides comprising canine CYP1A2 polypeptides and the polynucleotides that encode them. The invention also provides assays for screening compounds and therapeutics for metabolic responses indicative of a toxic compound or molecule.

### BACKGROUND OF THE INVENTION

The most well understood example in molecular toxicology is the induction of the microsomal cytochrome P450 dependent monooxygenases such as CYP1A1 (Cytochrome P450, subfamily I, member A1) and CYP1A2 (Cytochrome P450, subfamily I, member A2), that occurs in response to exposure to planar aromatic hydrocarbons (PAHs) or dioxins (1-3). These chemicals cause potent and pleiotropic toxicity, including teratogenesis, immune suppression, epithelial disorders, and tumor production in experimental animals. All these biologic effects are thought to be mediated by aryl hydrocarbon receptor (AHR).

The human CYP1A2 gene encodes the phenacetin O-deethylase also known as P450(PA) (4). Butler et al. reported in both mice and human the clearance of caffeine is determined primarily by CYP1A2 (4, 5).

Individuals with the most common form of porphyria, porphyria cutanea tarda (PCT, 176100), are believed to be genetically predisposed to development of clinically overt disease through mutations and polymorphisms in genes associated with known precipitating factors. Christiansen et al. (2000) examined a group of Danish patients with PCT for the presence of a C/A polymorphism in intron 1 of CYP1A2. The results demonstrated that the frequency of the highly inducible A/A genotype is increased in both familial and sporadic PCT. The authors suggested that the A/A genotype is a susceptibility factor for PCT.

To better understand the function of the CYP1A2 gene, a CYP1A2-deficient mouse line was developed by homologous recombination in embryonic stem cells (6). Although the knockout mice shows no developmental abnormalities, the treatment with zoxazolamine, a known CYP1A2 substrate, revealed dramatically lengthened paralysis times of the homozygous knockout mice relative to wildtype mice and the heterozygotes showed an intermediate effect, indicating CYP1A2 gene's role in drug metabolism.

Significant increases in the carcinogen-metabolizing enzymes CYP1A1, CYP1A2, CYP3A, CYP2B, and CYP2A were identified in the lungs of rats supplemented with high doses of beta-carotene. Paolini et al suggested that correspondingly high levels of CYPs in humans would predispose an individual to cancer risk from the widely bioactivated tobacco-smoke procarcinogens, thus explaining the cocarcinogenic effect of beta-carotene in smokers.

Because the dog is an important species for investigating the toxicology of drugs, nucleic acid probes specific for the canine CYP1A1 gene and antibodies specific for canine CYP1A1 protein have great value in industrial toxicology as a means to demonstrating a metabolic response to a suspected toxic compound. The present invention addresses this need.

### References Cited

1. Whitlock, J. P., Jr. Induction of cytochrome P4501A1. Ann. Rev. Pharmacol. Toxicol. 39:103-25, 1999.

2. Quattrochi, L. C., Vu, T., and Tukey, R. H. The human CYP1A2 gene and induction by 3-methylcholanthrene. J Biol. Chem. 269:6949-6954, 1994.

3. Li, W., Harper, P. A., Tang, B. K., and Okey, A. B. Regulation of cytochrome P450 enzymes by aryl hydrocarbon receptor in human cells: CYP1A2 expression in the LS180 colon carcinoma cell line after treatment with 2,3,7,8-tetrachlorodibenzo-p-dioxin or 3-methylcholanthrene. Biochm. Pharmacol. 56:599-612, 1998.

4. Butler, M. A., Iwasaki, M., Guengerich, F. P., Kadlubar, F. F. Human cytochrome P-450(PA) (P-450IA2), the phenacetin O-deethylase, is primarily responsible for the hepatic 3-demethylation of caffeine and N-oxidation of carcinogenic arylanines. Proc. Nat. Acad. Sci. 86: 7696-7700, 1989.

5. Buters, J. T. M., Tang, B.-K., Pineau, T., Gelboin, H. V., Kimura, S., Gonzalez, F. J. Role of CYP1A2 in caffeine pharmacokinetics and metabolism: studies using mice deficient in CYP1A2. Pharmacogenetics 6: 291-296, 1996.

6. Liang, H.-C. L., Li, H., McKinnon, R. A., Duffy, J. J., Potter, S. S., Puga, A., Nebert, D. W. Cyp1a2(−/−) null mutant mice develop normally but show deficient drug metabolism. Proc. Nat. Acad. Sci. 93: 1671-1676, 1996.

7. Paolini, M., Cantelli-Forti, G., Perocco, P., Pedulli, G. F., Abdel-Rahman, S. Z., Legator, M. S. Co-carcinogenic effect of beta-carotene. (Letter) Nature 398: 760-761, 1999.

### SUMMARY OF THE INVENTION

The present invention addresses the need identified above in that it provides isolated nucleic acid molecules encoding canine CYP1A2 protein, constructs and recombinant host cells incorporating the isolated nucleic acid molecules; the canine CYP1A2 polypeptide encoded by the isolated nucleic acid molecules; antibodies to the canine CYP1A2 polypeptide.

In one embodiment, the invention provides an isolated canine CYP1A2 polypeptide comprising the amino acid sequence set forth in SEQ ID NO: 2. It is understood that the polypeptide of SEQ ID NO:2 may be subject to specific proteolytic processing events resulting in a number of polypeptide species

In addition the invention provides a fragment comprising an epitope of the canine CYP1A2 polypeptide. By “epitope specific to” is meant a portion of the canine CYP1A2 polypeptide that is recognizable by an antibody that is specific for the canine CYP1A2 polypeptide, as defined in detail below. Another embodiment comprises an isolated polypeptide comprising the complete amino acid sequence set forth in SEQ ID NO: 2.

The coding cDNA sequence and predicted amino acid sequence of canine CYP1A2 is reproduced below and are also represented by SEQ ID NOS :1 and 2 respectively.

| SEQ ID NO:1 | | | | | | |
|---|---|---|---|---|---|---|
| 1 | atggcgttct | cccagtatat | ctccttagcc | ccagagctgc | tactggccac | tgccatcttc |
| 61 | tgtttagtgt | tctgggtgtt | gagaggcaca | aggacccagg | ttcccaaagg | tctgaagagt |
| 121 | cctcccggac | cctggggctt | gcccttcatt | gggcacatgc | tgaccctggg | gaagaaccca |
| 181 | cacctatctc | tgacaaagct | gagtcagcag | tatggggacg | tgctgcagat | ccgcattggc |
| 241 | tccacacccg | tggtggtgct | gagcggcctg | aacaccatca | agcaggccct | agtgaagcag |
| 301 | ggggatgact | tcaaaggccg | gccagacctc | tacagcttca | cacttatcac | taatggcaag |
| 361 | agcatgactt | tcaacccaga | ctctggaccg | gtctgggctg | cccgccggcg | cctggcccag |
| 421 | gatgccctga | agagtttctc | catagcctca | gaccccacat | cagtatcctc | ttgctacttg |
| 481 | gaggagcacg | tgagcaaaga | ggctaaccat | ctaatcagca | agttccagaa | gctgatggca |
| 541 | gaggttggcc | acttcgaacc | agtcaaccag | gtggtggaat | cggtggctaa | tgtcatcgga |
| 601 | gccatgtgtt | ttgggaagaa | cttccccagg | aagagcgagg | agatgctcaa | cctcgtgaag |
| 661 | agcagcaagg | actttgtgga | gaatgtcacc | tcagggaatg | ctgtggactt | ctttccggtc |
| 721 | ctgcgctacc | tgcccaaccc | agccctcaag | aggtttaaga | acttcaatga | taactttgtg |
| 781 | ctgtttctgc | agaaaacagt | ccaggaacac | tatcaagact | tcaacaagaa | cagtatccag |
| 841 | gacatcacag | gcgccctgtt | caagcacagt | gagaactaca | aagacaacgg | tggtctcatc |
| 901 | cctcaggaga | agattgtcaa | cattgtcaat | gacatctttg | gagctggatt | tgaaacagtc |
| 961 | acaacagcca | tcttctggag | cattttgcta | cttgtgacag | agcccaaggt | gcagaggaag |
| 1021 | attcatgagg | agctggacac | ggtgattggc | agagatcggc | agccacggct | ttctgacaga |
| 1081 | ccccagctgc | catatctgga | ggccttcatc | ctggagatct | accgatacac | atcctttgtc |
| 1141 | cccttcacca | tcccccacag | cacaacgagg | gacacctcac | tgaatggctt | ccacattccc |
| 1201 | aaggagtgct | gcatcttcat | aaaccagtgg | caggtcaacc | atgatgagaa | gcagtggaaa |
| 1261 | gacccctttg | tgttccgccc | agagcggttt | cttaccaatg | acaacacggc | catcgacaag |
| 1321 | accctgagtg | agaaggtgat | gctcttcggc | ttgggaaagc | gccggtgcat | tggggagatc |
| 1381 | ccggccaagt | gggaagtctt | cctcttctta | gccatcctcc | tgcatcagct | ggagttcact |
| 1441 | gtgccaccgg | gcgtgaaggt | ggacctgaca | cccagctatg | ggctgaccat | gaagcccaga |
| 1501 | acctgtgaac | acgtccaggc | ctggccacgc | ttctccaag | | |
| SEQ ID NO:2 | | | | | | |
| 1 | MAFSQYISLA | PELLLATAIF | CLVFWVLRGT | RTQVPKGLKS | PPGPWGLPFI | GHMLTLGKNP |
| 61 | HLSLTKLSQQ | YGDVLQIRIG | STPVVVLSGL | NTIKQALVKQ | GDDFKGRPDL | YSFTLITNGK |
| 121 | SMTFNPDSGP | VWAARRRLAQ | DALKSFSIAS | DPTSVSSCYL | EEHVSKEANH | LISKFQKLMA |
| 181 | EVGHFEPVNQ | VVESVANVIG | AMCFGKNFPR | KSEEMLNLVK | SSKDFVENVT | SGNAVDFFPV |
| 241 | LRYLPNPALK | RFKNFNDNFV | LFLQKTVQEH | YQDFNKNSIQ | DITGALFKHS | ENYKDNGGLI |
| 301 | PQEKIVNIVN | DIFGAGFETV | TTAIFWSILL | LVTEPKVQRK | IHEELDTVIG | RDRQPRLSDR |
| 361 | PQLPYLEAFI | LEIYRYTSFV | PFTIPHSTTR | DTSLNGFHIP | KECCIFINQW | QVNHDEKQWK |
| 421 | DPFVFRPERF | LTNDNTAIDK | TLSEKVMLFG | LGKRRCIGEI | PAKWEVFLFL | AILLHQLEFT |
| 481 | VPPGVKVDLT | PSYGLTMKPR | TCEHVQAWPR | FSK | | |

Although SEQ ID NOS: 1 and 2 provide particular canine polynucleotide and polypeptide sequences, the invention is intended to include within its scope other canine allelic variants.

In another embodiment, the invention provides isolated polynucleotides (e.g., cDNA, genomic DNA, synthetic DNA, RNA, or combinations thereof, single or double stranded) that comprise a nucleotide sequence encoding the amino acid sequence of the polypeptides of the invention. Such polynucleotides are useful for recombinantly expressing the enzyme and also for detecting expression of the enzyme in cells (e.g., using Northern hybridization and in situ hybridization assays). Such polynucleotides also are useful to design antisense and other molecules for the suppression of the expression of canine CYP1A2 in a cultured cell or tissue or in an animal, for therapeutic purposes or to provide a model for diseases characterized by aberrant canine CYP1A2 expression. Specifically excluded from the definition of polynucleotides of the invention are entire isolated chromosomes from native host cells from which the polynucleotide was originally derived. The polynucleotide set forth in SEQ ID NO: 1 corresponds to naturally occurring a canine CYP1A2 sequence. It will be appreciated that numerous other sequences exist that also encode the canine CYP1A2 of SEQ ID NO: 2 due to the well-known degeneracy of the universal genetic code. In another embodiment, the invention is directed to all of the degenerate canine CYP1A2 encoding sequences other than the sequence set forth in SEQ ID NO: 1.

The invention also provides an isolated polynucleotide comprising a nucleotide sequence, wherein the polynucleotide specifically hybridizes to the nucleotide sequence set forth in SEQ ID NO: 1 or the non-coding strand complementary thereto, under the following hybridization conditions:

(a) hybridization for 16 hours at 42° C. in a hybridization solution comprising 50% formamide, 1% SDS, 1M NaCl, 10% Dextran sulfate; and

(b) washing 2 times for 30 minutes at 60° C. in a wash solution comprising 0.1% SSC, 1% SDS.

One polynucleotide of the invention comprises the sequence set forth in SEQ ID NO: 1, which comprises a canine CYP1A2 encoding DNA sequence or unique fragments thereof.

In a related embodiment, the invention provides vectors comprising a polynucleotide of the invention. Such vectors are useful, e.g., for amplifying the polynucleotides in host cells to create useful quantities thereof. In other embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed or transfected (stably or transiently) with polynucleotides of the invention or vectors of the invention. As stated above, such host cells are useful for amplifying the polynucleotides and also for expressing the canine CYP1A2 polypeptide or fragment thereof encoded by the polynucleotide.

In still another related embodiment, the invention provides a method for producing a canine CYP1A2 polypeptide (or fragment thereof) comprising the steps of growing a host cell of the invention in a nutrient medium and isolating the polypeptide or variant thereof from the cell or the medium.

In still another embodiment, the invention provides an antibody that is specific for the canine CYP1A2 of the invention. Antibody specificity is described in greater detail below. However, it should be emphasized that antibodies that can be generated from polypeptides that have previously been described in the literature and that are capable of fortuitously cross-reacting with canine CYP1A2 (e.g., due to the fortuitous existence of a similar epitope in both polypeptides) are considered “cross-reactive” antibodies. Such cross-reactive antibodies are not antibodies that are “specific” for canine CYP1A2. The determination of whether an antibody is specific for canine CYP1A2 or is cross-reactive with another known enzyme is made using Western blotting assays or several other assays well known in the literature. For identifying cells that express canine CYP1A2 and also for modulating canine CYP1A2 activity, antibodies that specifically bind to the active site of canine CYP1A2 are particularly useful but of course, antibodies binding other epitopes are contemplated as part of the invention as well.

In one variation, the invention provides monoclonal antibodies. Hybridomas that produce such antibodies also are intended as aspects of the invention.

In another variation, the invention provides a cell-free composition comprising polyclonal antibodies, wherein at least one of the antibodies is an antibody of the invention specific for canine CYP1A2. Antiserum isolated from an animal is an exemplary composition, as is a composition comprising an antibody fraction of an antiserum that has been resuspended in water or in another diluent, excipient, or carrier.

The invention also provides methods of using antibodies of the invention. For example, the invention provides a method for determining the amount of canine CYP1A2 present within a cellular extract comprising the step of contacting canine CYP1A2 polypeptide with an antibody specific for the canine CYP1A2 polypeptide, under conditions wherein the antibody binds the canine CYP1A2 polypeptide.

The invention also provides a method for determining the amount of canine CYP1A2 polynucleotide present within a sample comprising: contacting the sample with a nucleic acid molecule comprising SEQ ID NO:1 or fragments or their complements thereof under conditions for the formation of one or more specific hybridization complexes, wherein the fragments are polynucleotides comprising at least 12 consecutive nucleotides of SEQ ID NO:1.

The invention also provides a method for measuring the metabolic response to a test agent in a dog comprising: providing a sample containing nucleic acids from a dog treated with a test agent; and determining the amount of polynucleotide comprising SEQ ID NO:1, or a fragment thereof or their complements in said sample, wherein a change in the amount of the polynucleotide from a treated dog, as compared with the amount of the polynucleotide from an untreated dog, is indicative of a metabolic response to the test agent.

In one embodiment the comparing comprises contacting nucleic acid molecules with the sample nucleic acid molecules under conditions effective to form hybridization complexes between nucleic acid molecules and the sample nucleic acid molecules; and detecting the presence or absence of the hybridization complexes. In one aspect, SEQ ID NO: 1 or fragments of SEQ ID NO:1 or complements of SEQ ID NO:1 may be present with a plurality of nucleic acids on a solid substrate array or other solid support.

The invention further provides a method for determining the amount of canine CYP1A2 polypeptide present within a sample comprising: contacting a canine CYP1A2 polypeptide with an antibody specific for the canine CYP1A2 polypeptide, under conditions wherein the antibody binds the canine CYP1A2 polypeptide.

The invention also provides a method for measuring the metabolic response to a test agent in a dog comprising: providing a sample from a dog treated with a test agent; and determining the amount of polypeptide comprising SEQ ID NO:2, or a fragment thereof comprising an epitope specific to said polypeptide in said sample, wherein a change in the amount of the polypeptide from a treated dog, as compared with the amount of the polypeptide from an untreated dog, is indicative of a metabolic response to the test agent.

### BRIEF DESCRIPTION OF THE SEQUENCE LISTINGS

SEQ ID NO: 1—cDNA sequence encoding canine CYP1A2

SEQ ID NO: 2—predicted amino acid sequence of canine CYP1A2

SEQ ID NOS: 3-8—cloning and sequencing primers

SEQ ID NO: 9 cDNA sequence encoding human CYP1A2

SEQ ID NO: 10 predicted amino acid sequence of human CYP1A2

SEQ ID NO: 11 cDNA sequence encoding mouse CYP1A2

SEQ ID NO: 12 predicted amino acid sequence of mouse CYP1A2

SEQ ID NO: 13 cDNA sequence encoding rat CYP1A2

SEQ ID NO: 14 predicted amino acid sequence of rat CYP1A2

### BRIEF DESCRIPTION OF THE DRAWINGS

FIG. 1 Alignment of Dog (SEQ ID NO: 1), Human (SEQ ID NO: 10) Mouse (SEQ ID NO: 12) and Rat (SEQ ID NO: 14) CYP1A2 polypeptide sequences

FIG. 2 Alignment of Dog (SEQ ID NO: 2), Human (SEQ ID NO: 9) Mouse (SEQ ID NO: 11) and Rat (SEQ ID NO: 13) CYP1A2 polynucleotide sequences

### DETAILED DESCRIPTION OF THE INVENTION

General Definitions

As used hereinafter “polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, aswell as the chemical forms of DNA and RNA characteristic of viruses and cells. “polynucleotide” also embraces relatively short polynucleotides, often referred to as oligonucleotides.

As used hereinafter “polypeptide” refers to any peptide or protein comprising amino acids joined to each other by peptide bonds or modified peptide bonds. “polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications or modified forms include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Postranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:4842).

“Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

As used hereinafter “isolated” means altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein. “Isolated” as used herein and as understood in the art, whether referring to “isolated” polynucleotides or polypeptides, is taken to mean separated from the original cellular environment in which the polypeptide or nucleic acid is normally found. As used herein therefore, by way of example only, a transgenic animal or a recombinant cell line constructed with a polynucleotide of the invention, makes use of the “isolated” nucleic acid.

As used herein, the term “canine CYP1A2 polypeptide” means a protein encoded by a canine CYP1A2 gene, including allelic variants containing conservative or non-conservative changes. One canine CYP1A2 protein sequence is disclosed as SEQ ID NO:2.

The canine CYP1A2 polypeptide may be produced by recombinant cells or organisms, may be substantially purified from natural tissues or cell lines, or may be synthesized chemically or enzymatically. Therefore, the term “canine CYP1A2 polypeptide is intended to include the protein in glycosylated, partially glycosylated, or unglycosylated forms, as well as in phosphorylated, partially phosphorylated, unphosphorylated, sulphated, partially sulphated, or unsulphated forms. The term also includes allelic variants, other functional equivalents of the PS2 amino acid sequence, including biologically active proteolytic or other fragments, and physiological and pathological proteolytic cleavage products of the canine CYP1A2 polypeptide.

As used herein, the term “test agent” means any identifiable chemical or molecule, including, but not limited to a small molecule, peptide, protein, sugar, nucleotide, or nucleic acid. Such a test agent can be natural or synthetic.

As used herein, the term “contacting” means bringing together, either directly or indirectly, a compound into physical proximity to a polypeptide or polynucleotide of the invention. The polypeptide or polynucleotide can be present in any number of buffers, salts, solutions, etc. Contacting includes, for example, placing the compound into a beaker, microtiter plate, cell culture flask, or a microarray, such as a gene chip, or the like, which contains either the ion channel polypeptide or fragment thereof, or nucleic acid molecule encoding an ion channel or fragment thereof.

Nucleic Acids of the Invention

The present invention provides isolated polynucleotides (e.g., DNA sequences and RNA transcripts, both sense and complementary antisense strands, both single and double-stranded, including splice variants thereof) encoding a canine CYP1A2 polypeptide referred to herein as canine CYP1A2. DNA polynucleotides of the invention include genomic DNA, cDNA, and DNA that has been chemically synthesized in whole or in part.

Genomic DNA of the invention comprises the protein coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a canine CYP1A2 polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation).

The invention also comprehends cDNA that is obtained through reverse transcription of an RNA polynucleotide encoding canine CYP1A2 (conventionally followed by second strand synthesis of a complementary strand to provide a double-stranded DNA).

A DNA sequence encoding a canine CYP1A2 polypeptide is set out in SEQ ID NO: 1. The worker of skill in the art will readily appreciate that the DNA of the invention comprises a double stranded molecule, for example the molecule having the sequence set forth in SEQ ID NO: 1 along with the complementary molecule (the “non-coding strand” or “complement”) having a sequence deducible from the sequence of SEQ ID NO: 1 according to Watson-Crick base pairing rules for DNA. Also contemplated by the invention are other polynucleotides encoding the canine CYP1A2 polypeptide of SEQ ID NO: 2, which differ in sequence from the polynucleotide of SEQ ID NO: 1 by virtue of the well-known degeneracy of the universal genetic code.

As is well known in the art, due to the degeneracy of the genetic code, there are numerous other DNA and RNA molecules that can code for the same polypeptide as that encoded by the aforementioned SEQ ID NO: 1 polypeptides. The present invention, therefore, contemplates those other DNA and RNA molecules which, on expression, encode the polypeptides of SEQ ID NO: 2 Having identified the amino acid residue sequence encoded the canine CYP1A2 polypeptide, and with the knowledge of all triplet codons for each particular amino acid residue, it is possible to describe all such encoding RNA and DNA sequences. DNA and RNA molecules other than those specifically disclosed herein characterized simply by a change in a codon for a particular amino acid, are, therefore, within the scope of this invention.

A table of amino acids and their representative abbreviations, symbols and codons is set forth below in the following Table 1.

TABLE 1

| | | | |
|---|---|---|---|
| Amino acid | Abbrev. | Symbol | Codon(s) |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

As is well known in the art, codons constitute triplet sequences of nucleotides in mRNA and their corresponding cDNA molecules Codons are characterized by the base uracil (U) when present in a mRNA molecule but are characterized by base thymidine (T) when present in DNA. A simple change in a codon for the same amino acid residue within a polynucleotide will not change the sequence or structure of the encoded polypeptide. It is apparent that when a phrase stating that a particular 3 nucleotide sequence “encode(s)” any particular amino acid, the ordinarily skilled artisan would recognize that the table above provides a means of identifying the particular nucleotides at issue. By way of example, if a particular three nucleotide sequence encodes theonine the table above discloses that the posible triplet sequences are ACA, ACG, ACC and ACU (ACT if in DNA).

The invention further embraces species, preferably mammalian, homologs of the canine CYP1A2 DNA. Species homologs, sometimes referred to as “orthologs,” share at least 99.8%, 99.9% homology with SEQ ID NO: 1 of the invention. Percent sequence “homology” with respect to polynucleotides of the invention is defined herein as the percentage of nucleotide bases in the candidate sequence that are identical to nucleotides in the canine CYP1A2 sequence set forth in SEQ ID NO: 1, after aligning the sequences and introducing gaps, if necessary, to achieve the maximum percent sequence identity. The percentage of sequence between a native and a variant canine CYP1A2 sequence may also be determined, for example, by comparing the two sequences using any of the computer programs commonly employed for this purpose, such as the Gap program (Wisconsin Sequence Analysis Package, Version 8 for Unix, Genetics Computer Group, University Research Park, Madison Wis.), which uses the algorithm of Smith and Waterman (Adv. Appl. Math. 2: 482-489 (1981)).

The polynucleotide sequence information provided by the invention makes possible large scale expression of the encoded polypeptide by techniques well known and routinely practiced in the art. Polynucleotides of the invention also permit identification and isolation of polynucleotides encoding related canine CYP1A2 polypeptides, such as human allelic variants and species homologs, by well known techniques including Southern and/or Northern hybridization, and polymerase chain reaction (PCR). Examples of related polynucleotides include human and non-human genomic sequences, including allelic variants, as well as polynucleotides encoding polypeptides homologous to canine CYP1A2 and structurally related polypeptides sharing one or more biological, immunological, and/or physical properties of canine CYP1A2. Knowledge of the sequence of a canine CYP1A2 DNA also makes possible through use of Southern hybridization or polymerase chain reaction (PCR) the identification of genomic DNA sequences encoding canine CYP1A2 expression control regulatory sequences such as promoters, operators, enhancers, repressors, and the like. Polynucleotides of the invention are also useful in hybridization assays to detect the capacity of cells to express canine CYP1A2 or to measure levels of canine CYP1A2 expression. Polynucleotides of the invention may also be the basis for diagnostic methods useful for identifying a genetic alteration(s) in a canine CYP1A2 locus that underlies a disease state or states, which information is useful both for diagnosis and for selection of therapeutic strategies.

The disclosure herein of a full length polynucleotide encoding a canine CYP1A2 polypeptide makes readily available to the worker of ordinary skill in the art every possible fragment of the full length polynucleotide. The invention therefore provides fragments of canine CYP1A2-encoding polynucleotides comprising at least 12 through 2562 (including each and every integer value between) consecutive nucleotides of a polynucleotide encoding canine CYP1A2. Polynucleotides of the invention (including fragments) often comprise sequences unique to the canine CYP1A2-encoding polynucleotide sequence, and therefore would hybridize under highly stringent or moderately stringent conditions only (i.e., “specifically”) to polynucleotides encoding canine CYP1A2 (or fragments thereof). Polynucleotide fragments of genomic sequences of the invention comprise not only sequences unique to the coding region, but also include fragments of the full length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern hybridization analyses to determine the number of fragments of genomic DNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full length or other fragment canine CYP1A2 polynucleotides. One or more fragment polynucleotides can be included in kits that are used to detect the presence of a polynucleotide encoding canine CYP1A2, or used to detect variations in a polynucleotide sequence encoding canine CYP1A2.

The invention also embraces DNAs encoding canine CYP1A2 polypeptides which DNAs hybridize under high stringency hybridization or wash conditions to the non-coding strand, or complement, of the polynucleotide in SEQ ID NO: 1.

The concept of high stringency hybridization is discussed below in the section detailing the “Assays of the Invention”.

### EXAMPLE 1

### Cloning of Canine CYP1A2

To design primers for PCR, the open reading frame (ORF) sequences of each gene for human, rat and mouse CYP1A2 were aligned using the DNASTAR software (DNASTAR, WI). Three sets of primers were chosen for each gene. Among them, 2 sets correspond to the start and stop sequence regions of the human and the rat orthologs respectively; the other set was designed according to the conserved sequence regions among human, rat and mouse orthologs. The primers are generally 30 nucleotides in length.

The tissue expression pattern of the picked cloning candidate genes were identified through HumanPSD database (Proteome Inc.) and through NCBI EST database search (by identifying the tissue from which the homologous ESTs were cloned). It was identified that 5 tissues, liver, kidney, colon, spleen and lung, covered all 12 picked genes.

The RNA from a canine liver sample was prepared with the Rneasy kit (Qiagen, CA). To reverse transcribe RNA into cDNA, 0.5 ug of RNA was mixed with 5.77 ul of 32.5 uM Random Hexamers (Amersham Biosciences, NJ) and 2.5 ul of 10 mM dNTP in a total volume of 12.5 ul. The mixture was incubated at 65° C. for 5 min and chilled on ice. A 12.5 ul master mix containing 2 ul of RNase-free water, 5 ul of 5×1st strand buffer (Invitrogen, CA), 0.5 ul of 100 mM DTT, 2.5 ul of 40 U/ul RNaseOUT (Invitrogen, CA) and 2.5 ul of 200 U/ul Superscript II reverse transcriptase (Invitrogen, CA) was added. The final reaction mixture was incubated consecutively at 25° C. for 10 minutes, 42° C. for 60 minutes and 70° C. for 15 minutes. The synthesized cDNA was diluted 20 fold (final concentration around 1 ng/ul) and stored at −20° C. freezers for direct use as templates for PCR reactions.

Modified PCR reactions with conditions adapted from RACE (Rapid Amplification of cDNA ends) reactions were used to clone canine genes or gene fragments. For a 50 ul PCR reaction, 5 ul of reverse transcribed cDNA, 5 ul 10×PCR buffer (Clontech, CA), 1 ul dNTPs of the 50× dNTP mix (10 mM each, final 0.2 mM each, Clontech, CA), 1 ul of 5′ and 3′ primer each at 20 uM. The primer sequences used were:

| | |
|---|---|
| ATGGCGTTCTCCCAGTATATCTCCTTAGCC | (SEQ ID NO:3) |
| TCACTTGGAGAAGCGTGGCCAGGCCTGGAC | (SEQ ID NO:4) |

1 ul of Clontech Advantage 2 Taq polymerase (50×) and 36 ul PCR water (Clontech, CA) were mixed. The mixture was incubated at 94° C. for 1 minute and going through a touch down PCR protocol for 5 cycles at 94° C. for 15 seconds and then to 72° C. 4 minutes, 5 cycles at 94° C. for 15 seconds and then to 70° C. for 4 minutes, 25 cycles at 94° C. for 15 seconds and then to 68° C. for 4 minutes. For genes that are not cloned from the first round of PCR reaction, the less stringent PCR conditions are 5 cycles at 94° C. for 15 seconds and then to 68° C. 4 minutes, 5 cycles at 94° C. for 15 seconds and then to 65° C. for 4 minutes, 25 cycles at 94° C. for 15 seconds and then to 62° C. for 4 minutes. The length and concentration of the PCR fragments are examined on Agilent 2100 Bioanalyzer using DNA 7500 chip (Agilent, CA) according to manufacture's instructions.

DNA Preparation and Sequencing

The PCR product was sequenced using an ABI377 fluorescence-based sequencer (Perkin Elmer/Applied Biosystems Division, PE/ABD, Foster City, Calif.) and the ABI PRISMTM Ready Dye-Deoxy Terminator kit with Taq FSTM polymerase with the following primers:

| | |
|---|---|
| CTGCTTCACTAGGGCCTGCTTG | (SEQ ID NO:5) |
| GCAAAGAGGCTAACCATCTAATC | (SEQ ID NO:6) |
| GCAAAATGCTCCAGAAGATGGCTG | (SEQ ID NO:7) |
| GGCCTTCATCCTGGAGATCTAC | (SEQ ID NO:8) |

Each ABI cycle sequencing reaction contains about 0.5 μg of plasmid DNA. Cycle-sequencing is performed using an initial denaturation at 98° C. for 1 minute, followed by 50 cycles: 98° C. denaturation for 30 seconds, annealing at 50° C. for 30 seconds, and extension at 60° C. for 4 minutes. Temperature cycles and times are controlled by a Perkin-Elmer 9600 thermocycler. Extension products are isolated using CentriflexTM gel filtration cartridges (Advanced Genetic Technologies Corp., Gaithersburg, Md.). Each reaction product is loaded by pipette onto the column, which is then centrifuged in a swinging bucket centrifuge (Sorvall model RT6000B table top centrifuge) at 1500×g for 4 minutes at room temperature. Column-purified samples are dried under a vacuum for about 40 minutes and then dissolved in 5 μl of a DNA loading solution (83% deionized formamide, 8.3 mM EDTA, and 1.6 mg/ml Blue Dextran). The samples are then heated to 90° C. for three minutes and loaded into the gel sample wells for sequence analysis by the ABI377 sequencer. Sequence analysis was done by importing ABI377 files into the DNASTAR (DNASTAR Madison, Wis.) program. Generally sequence reads of 700 bp were obtained. Potential sequence errors were minimized by obtaining sequence information from both DNA strands and by re-sequencing difficult areas using primers at different locations until all sequencing ambiguities are removed. SEQ ID NO:1 represents the correct sequence of the product.

The sequencing data were aligned with human gene orthologs and curated manually according to sequence trace data. For cloned genes or gene fragment that are not completely finished after first round of sequencing, more sequencing primers were ordered based on the curated sequence data until the whole project was completed. The dog coding sequence encodes a protein which is 74%, 99% and 93% identical to the human, rat and mouse homologs respectively. The dog DNA coding sequence is 80.4%, 99.7 %, 93.5% identical respectively to the human, rat and mouse coding DNA sequences. (Comparisons based on Genbank NM_{—}000761 for human, NM_{—}009993 for mouse and NM_{—}012541 for rat mRNA NP_{—}000752 for human, NP_{—}034123 for mouse and NP_{—}036673 for rat protein).

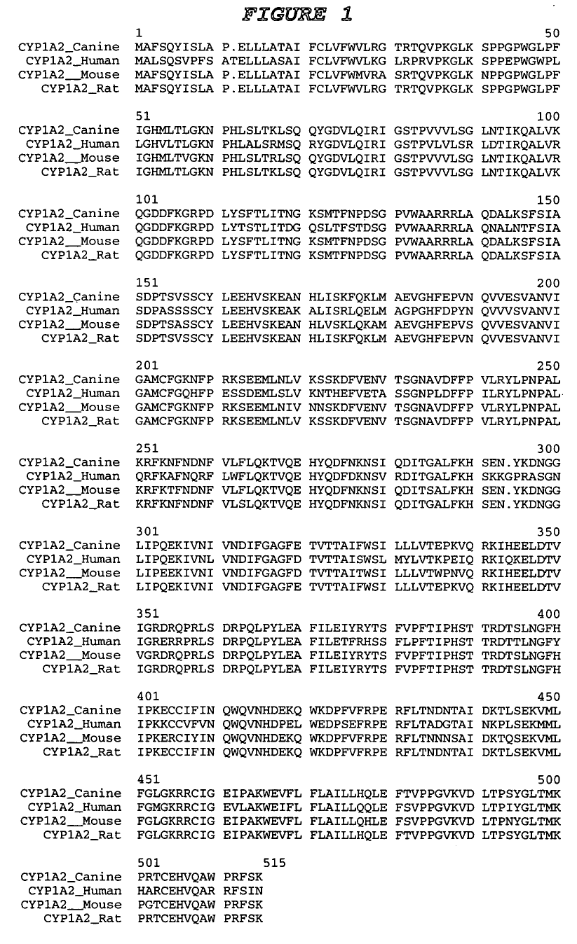

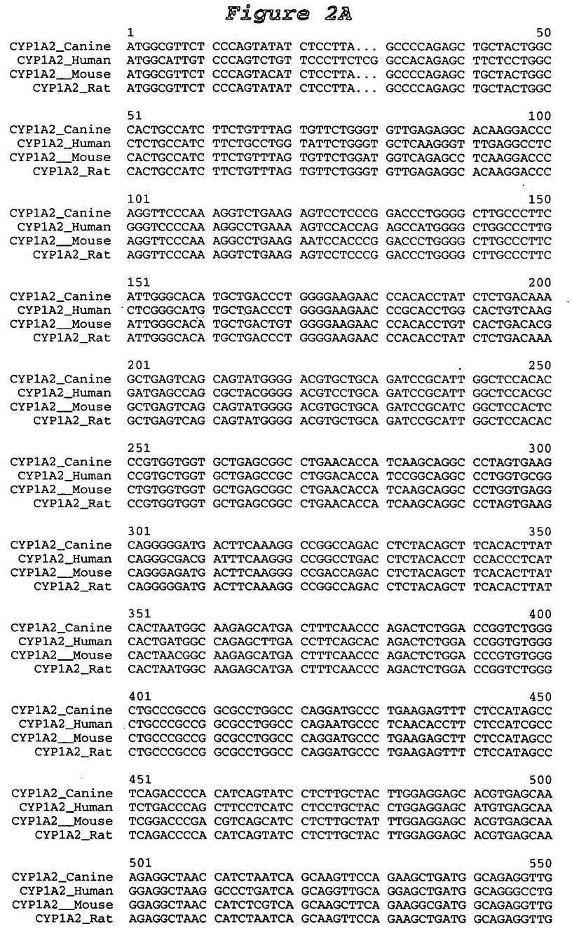

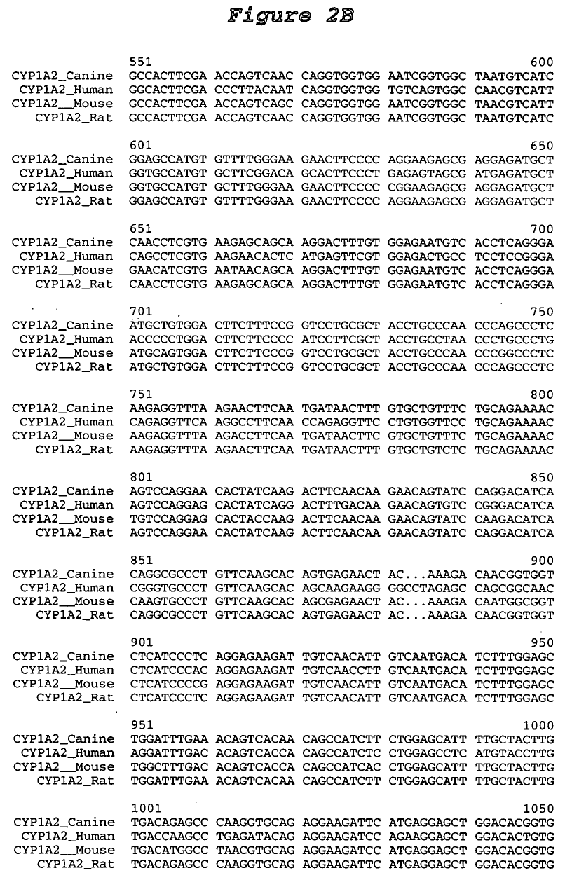

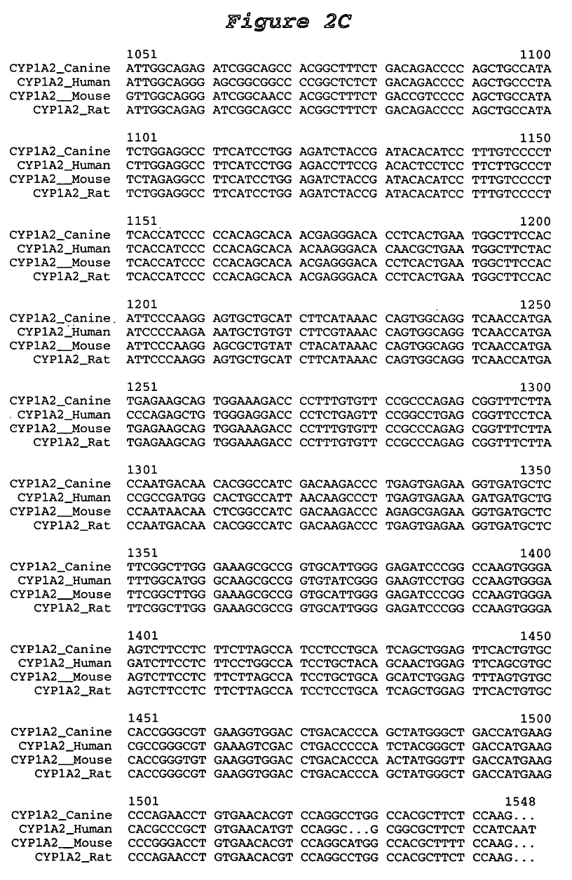

Alignments of the newly discovered dog sequence with the other three homologues is shown in FIGS. 1 and 2.

It should be recognized that this method of obtaining the sequence of SEQ ID NO:1 or exemplary and that by disclosing SEQ ID NO:1 it provides one skilled in the art a multitude of methods of obtaining the entire sequence of SEQ ID NO:1. By way of example, it would be possible to generate probes from the sequence disclosed in SEQ ID NO:1 and screen dog cDNA or genomic libraries and thereby obtain the entire SEQ ID NO:1 or its genomic equivalent. Sambrook, et al., (Eds.), Molecular Cloning: A Laboratory Manual, Cold Spring Harbor Laboratory Press: Cold Spring Harbor, N.Y. (1989). Also by way of example, one skilled in the art would immediately recognize that given the sequence disclosed in SEQ ID NO:1 it is then possible to generate the appropriate primers for PCR amplification to obtain the entire sequence represented by SEQ ID NO:1. (see e.g., PCR Technology, H. A. Erlich, ed., Stockton Press, New York, 1989; PCR Protocols: A Guide to Methods and Applications, M. A. Innis, David H. Gelfand, John J. Sninsky, and Thomas J. White, eds., Academic Press, Inc., New York, 1990.

Host Cells and Vectors of the Invention

Autonomously replicating recombinant expression constructs such as plasmid and viral DNA vectors incorporating polynucleotides of the invention are also provided. Expression constructs wherein canine CYP1A2-encoding polynucleotides are operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Constructs of the invention also include sequences necessary for replication in a host cell.

Expression constructs are preferably utilized for production of an encoded protein, but also may be utilized simply to amplify a canine CYP1A2 encoding polynucleotide sequence.

According to another aspect of the invention, host cells are provided, including prokaryotic and eukaryotic cells, comprising a polynucleotide of the invention (or vector of the invention) in a manner which permits expression of the encoded canine CYP1A2 polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Suitable host cells for expression of canine CYP1A2 polypeptides include prokaryotes, yeast, and higher eukaryotic cells. Suitable prokaryotic hosts to be used for the expression of canine CYP1A2 polypeptides include but are not limited to bacteria of the genera Escherichia, Bacillus, and Salmonella, as well as members of the genera Pseudomonas, Streptomyces, and Staphylococcus.

The isolated nucleic acid molecules of the invention are preferably cloned into a vector designed for expression in eukaryotic cells, rather than into a vector designed for expression in prokaryotic cells. Eukaryotic cells are sometimes preferred for expression of genes obtained from higher eukaryotes because the signals for synthesis, processing, and secretion of these proteins are usually recognized, whereas this is often not true for prokaryotic hosts (Ausubel, et al., ed., in Short Protocols in Molecular Biology, 2nd edition, John Wiley & Sons, publishers, pg.16-49, 1992.). In the case of the canine CYP1A2, there are 2 consensus sequences for N-linked glycosylation, and other sites of post-translational modification can be predicted for protein kinase C phosphorylation and O-glycosylation. Eukaryotic hosts may include, but are not limited to, the following: insect cells, African green monkey kidney cells (COS cells), Chinese hamster ovary cells (CHO cells), human 293 cells, and murine 3T3 fibroblasts.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), and pQE vectors (Qiagen).

Canine CYP1A2 may also be expressed in yeast host cells from genera including Saccharomyces, Pichia, and Kluveromyces. Yeast hosts include S. cerevisiae and P. pastoris. Yeast vectors will often contain an origin of replication sequence from a 2 micron yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of canine CYP1A2 polypeptides expressed in yeast hosts may be accomplished by the inclusion of nucleotide sequence encoding the yeast factor leader sequence at the 5′ end of the canine CYP1A2-encoding nucleotide sequence.

Insect host cell culture systems may also be used for the expression of canine CYP1A2 polypeptides. In another embodiment, the canine CYP1A2 polypeptides of the invention are expressed using a baculovirus expression system. Further information regarding the use of baculovirus systems for the expression of heterologous proteins in insect cells are reviewed by Luckow and Summers, Bio/Technology 6:47 (1988).

In another embodiment, the canine CYP1A2 polypeptide is expressed in mammalian host cells. Non-limiting examples of suitable mammalian cell lines include the COS-7 line of monkey kidney cells (Gluzman et al., Cell 23:175 (1981)), Chinese hamster ovary (CHO) cells, and human 293 cells.

The choice of a suitable expression vector for expression of the canine CYP1A2 polypeptides of the invention will of course depend upon the specific host cell to be used, and is within the skill of the ordinary artisan. Examples of suitable expression vectors include pcDNA3 (Invitrogen) and pSVL (Pharmacia Biotech). Expression vectors for use in mammalian host cells may include transcriptional and translational control sequences derived from viral genomes. Commonly used promoter sequences and modifier sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

### EXAMPLE 2

### Expression f Canine CYP1A2 in Eukaryotic Host Cells

To produce canine CYP1A2 protein, a canine CYP1A2-encoding polynucleotide is expressed in a suitable host cell using a suitable expression vector, using standard genetic engineering techniques. For example, the canine CYP1A2-encoding sequences described in Example 1 are subcloned into the commercial expression vector pzeoSV2 (Invitrogen, San Diego, Calif.) and transfected into Chinese Hamster Ovary (CHO) cells using the transfection reagent fuGENE 6 (Boehringer-Mannheim) and the transfection protocol provided in the product insert. Other eukcryotic cell lines, including human embryonic kidney HEK 293 and COS cells, are suitable as well. Cells stably expressing canine CYP1A2 are selected by growth in the presence of 100 μg/ml zeocin (Stratagene, LaJolla, Calif.). Optionally, the canine CYP1A2 is isolated from the cells using standard chromatographic techniques. To facilitate purification, antisera may be raised against one or more synthetic peptide sequences that correspond to portions of the canine CYP1A2 amino acid sequence, and the antisera is used to affinity purify canine CYP1A2. The canine CYP1A2 also may be expressed in frame with a tag sequence (e.g., polyhistidine, hemaggluttinin, FLAG) to facilitate purification.

Host cells of the invention are a valuable source of immunogen for development of antibodies specifically immunoreactive with canine CYP1A2. Host cells of the invention are also useful in methods for large scale production of canine CYP1A2 polypeptides wherein the cells are grown in a suitable culture medium and the desired polypeptide products are isolated from the cells or from the medium in which the cells are grown by purification methods known in the art, e.g., conventional chromatographic methods including immunoaffinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and isolated as a fusion protein having a specific tag, label, or chelating moiety that is recognized by a specific binding partner or agent. The isolated protein can be cleaved to yield the desired protein, or be left as an intact fusion protein. Cleavage of the fusion component may produce a form of the desired protein having additional amino acid residues as a result of the cleavage process.

Knowledge of canine CYP1A2 DNA sequences allows for modification of cells to permit, or increase, expression of endogenous canine CYP1A2. Cells can be modified (e.g., by homologous recombination) to provide increased expression by replacing, in whole or in part, the naturally occurring canine CYP1A2 promoter with all or part of a heterologous promoter so that the cells express canine CYP1A2 at higher levels. The heterologous promoter is inserted in such a manner that it is operatively linked to endogenous canine CYP1A2 encoding sequences. [See, for example, PCT International Publication No. WO 94/12650, PCT International Publication No. WO 92/20808, and PCT International Publication No. WO 91/09955.] It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the canine CYP1A2 coding sequence, amplification of the marker DNA by standard selection methods results in co-amplification of the canine CYP1A2 coding sequences in the cells.

The DNA sequence information provided by the present invention also makes possible the development through, e.g. homologous recombination or “knock-out” strategies [Capecchi, Science 244:1288-1292 (1989)], of animals that fail to express functional canine CYP1A2 or that express a variant of canine CYP1A2. Such animals (especially small laboratory animals such as rats, rabbits, and mice) are useful as models for studying the in vivo activities of canine CYP1A2 and modulators of canine CYP1A2.

Also made available by the invention are anti-sense polynucleotides which recognize and hybridize to polynucleotides encoding canine CYP1A2. Full length and fragment anti-sense polynucleotides are provided. Fragment anti-sense molecules of the invention include (i) those which specifically recognize and hybridize to canine CYP1A2 (as determined by sequence comparison of DNA encoding canine CYP1A2 to DNA encoding other known molecules). Identification of sequences unique to canine CYP1A2-encoding polynucleotides, can be deduced through use of any publicly available sequence database, and/or through use of commercially available sequence comparison programs. The uniqueness of selected sequences in an entire genome can be further verified by hybridization analyses. After identification of the desired sequences, isolation through restriction digestion or amplification using any of the various polymerase chain reaction techniques well known in the art can be performed. Anti-sense polynucleotides are particularly relevant to regulating expression of canine CYP1A2 by those cells expressing canine CYP1A2 mRNA.

Antisense nucleic acids (preferably 10 to 20 base pair oligonucleotides) capable of specifically binding to canine CYP1A2 expression control sequences or canine CYP1A2 RNA are introduced into cells (e.g., by a viral vector or colloidal dispersion system such as a liposome). The antisense nucleic acid binds to the canine CYP1A2 target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of canine CYP1A2 expression at either the transcriptional or translational level is useful to generate cellular or animal models for diseases characterized by aberrant canine CYP1A2 expression or as a therapeutic modality.

The canine CYP1A2 sequences taught in the present invention facilitate the design of novel transcription factors for modulating canine CYP1A2 expression in native cells and animals, and cells transformed or transfected with canine CYP1A2 polynucleotides. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular canine CYP1A2 target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on canine CYP1A2 sequences, are fused to activation or repression domains to promote or suppress canine CYP1A2 expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating canine CYP1A2 expression in vivo in both native cells, animals and humans; and/or cells transfected with canine CYP1A2-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348]. The present invention contemplates methods of designing such transcription factors based on the gene sequence of the invention, as well as customized zinc finger proteins, that are useful to modulate canine CYP1A2 expression in cells (native or transformed) whose genetic complement includes these sequences.

The invention also provides isolated mammalian canine CYP1A2 polypeptides encoded by a polynucleotide of the invention.

Polypeptides of the Invention

The canine CYP1A2 polypeptide amino acid sequence is set out in SEQ ID NO: 2. The amino acid sequence of the invention as exemplified by SEQ ID NO:2 has several interesting features.

The invention also embraces polypeptides that have greater than 99% identity and/or homology to the polypeptide polypeptide set out in SEQ ID NO: 2. Percent amino acid sequence “identity” with respect to the polypeptide of SEQ ID NO: 2 is defined herein as the percentage of amino acid residues in the candidate sequence that are identical with the residues in the canine CYP1A2 sequence after aligning both sequences and introducing gaps, if necessary, to achieve the maximum percent sequence identity, and not considering any conservative substitutions as part of the sequence identity. Percent sequence “homology” with respect to the polypeptide of SEQ ID NO: 2 is defined herein as the percentage of amino acid residues in the candidate sequence that are identical with the residues in the canine CYP1A2 sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment [Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated form of canine CYP1A2 polypeptides are embraced.

Overexpression in eukaryotic and prokaryotic hosts as described above facilitates the isolation of canine CYP1A2 polypeptides. The invention therefore includes isolated canine CYP1A2 polypeptides as set out in SEQ ID NO:2 and variants and conservative amino acid substitutions therein including labeled and tagged polypeptides.

The invention includes canine CYP1A2 polypeptides which are “labeled”. The term “labeled” is used herein to refer to the conjugating or covalent bonding of any suitable detectable group, including enzymes (e.g., horseradish peroxidase, beta-glucuronidase, alkaline phosphatase, and beta-D-galactosidase), fluorescent labels (e.g., fluorescein, luciferase), and radiolabels (e.g., ¹⁴C, ¹²⁵I, ³H, ³²P, and ³⁵S) to the compound being labeled. Techniques for labeling various compounds, including proteins, peptides, and antibodies, are well known. See, e.g., Morrison, Methods in Enzymology 32b, 103 (1974); Syvanen et al., J. Biol. Chem. 284, 3762 (1973); Bolton and Hunter, Biochem. J. 133, 529 (1973). The termed labeled may also encompass a polypeptide which has covalently attached an amino acid tag as discussed below.

In addition, the canine CYP1A2 polypeptides of the invention may be indirectly labeled. This involves the covalent addition of a moiety to the polypeptide and subsequent coupling of the added moiety to a label or labeled compound which exhibits specific binding to the added moiety. Possibilities for indirect labeling include biotinylation of the peptide followed by binding to avidin coupled to one of the above label groups. Another example would be incubating a radiolabeled antibody specific for a histidine tag with a canine CYP1A2 polypeptide comprising a polyhistidine tag. The net effect is to bind the radioactive antibody to the polypeptide because of the considerable affinity of the antibody for the tag.

The invention also embraces variants (or analogs) of the canine CYP1A2 protein. In one example, insertion variants are provided wherein one or more amino acid residues supplement a canine CYP1A2 amino acid sequence. Insertions may be located at either or both termini of the protein, or may be positioned within internal regions of the canine CYP1A2 protein amino acid sequence. Insertional variants with additional residues at either or both termini can include for example, fusion proteins and proteins including amino acid tags or labels. Insertion variants include canine CYP1A2 polypeptides wherein one or more amino acid residues are added to a canine CYP1A2 acid sequence, or to a biologically active fragment thereof.

Insertional variants therefore can also include fusion proteins wherein the amino and/or carboxy termini of canine CYP1A2 is fused to another polypeptide. Various tag polypeptides and their respective antibodies are well known in the art. Examples include poly-histidine (poly-his) or poly-histidine-glycine (poly-his-gly) tags; the influenza HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397(1990)]. In addition, the canine CYP1A2 polypeptide can be tagged with enzymatic proteins such as peroxidase and alkaline phosphatase.

In another aspect, the invention provides deletion variants wherein one or more amino acid residues in a canine CYP1A2 polypeptide are removed. Deletions can be effected at one or both termini of the canine CYP1A2 polypeptide, or with removal of one or more residues within the canine CYP1A2 amino acid sequence. Deletion variants, therefore, include all fragments of the canine CYP1A2 polypeptide.

The invention also embraces polypeptide fragments of the sequence set out in SEQ ID NO: 2 wherein the fragments maintain biological (e.g., ligand binding or DNA binding and/or other biological activity) Fragments comprising at least 10 through 853 (including each and every integer value between) consecutive amino acids of SEQ ID NO: 2 are comprehended by the invention. Fragments of the invention having the desired biological properties can be prepared by any of the methods well known and routinely practiced in the art.

The present invention also includes include variants of the aforementioned polypetide, that is polypeptides that vary from the reference sequence by conservative amino acid substitutions, whereby a residue is substituted by another with like characteristics. Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 2 (from WO 97/09433, page 10, published Mar. 13, 1997 (PCT/GB96/02197, filed Sep. 6, 1996), immediately below.

TABLE 2
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY:NY (1975), pp.71-77] as set out in Table 3, immediately below.

TABLE 3
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | DE |

As still another alternative, exemplary conservative substitutions are set out in Table 4, immediately below.

TABLE 4
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Generally it is anticipated that the canine CYP1A2 polypeptide will be found primarily intracellularly, the intracellular material can be extracted from the host cell using any standard technique known to the skilled artisan. For example, the host cells can be lysed to release the contents of the cytoplasm by homogenization, and/or sonication followed by centrifugation. The canine CYP1A2 polypeptide is found primarily in the supernatant after centrifugation of the cell homogenate, and the canine CYP1A2 polypeptide can be isolated by way of non-limiting example by any of the methods below.

In those situations where it is preferable to partially or completely isolate the canine CYP1A2 polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Purification of canine CYP1A2 polypeptide can be accomplished using a variety of techniques. If the polypeptide has been synthesized such that it contains a tag such as Hexahistidine (canine CYP1A2/hexaHis) or other small peptide such as FLAG (Eastman Kodak Co., New Haven, Conn.) or myc (Invitrogen, Carlsbad, Calif.) at either its carboxyl or amino terminus, it may essentially be purified in a one-step process by passing the solution through an affinity column where the column matrix has a high affinity for the tag or for the polypeptide directly (i.e., a monoclonal antibody specifically recognizing canine CYP1A2). For example, polyhistidine binds with great affinity and specificity to nickel, thus an affinity column of nickel (such as the Qiagen Registered TM nickel columns) can be used for purification of canine CYP1A2/polyHis. (See for example, Ausubel et al., eds., Current Protocols in Molecular Biology, Section 10.11.8, John Wiley & Sons, New York [1993]).

Even if the canine CYP1A2 polypeptide is prepared without a label or tag to facilitate purification. The canine CYP1A2 of the invention may be purified by immunoaffinity chromatography. To accomplish this, antibodies specific for the canine CYP1A2 polypeptide must be prepared by means well known in the art. Antibodies generated against the canine CYP1A2 polypeptides of the invention can be obtained by administering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in Monoclonal Antibodies and Cancer Therapy, Alan R. Liss, Inc. (1985).

Where the canine CYP1A2 polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. A representative purification scheme is detailed below.

Variants that display inhibitory properties of native canine CYP1A2 and are expressed at higher levels and variants that provide for constitutive active canine CYP1A2 polypeptide are particularly useful in assays of the invention and also useful in cellular and animal models for diseases characterized by aberrant canine CYP1A2 expression activity.

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for canine CYP1A2 or fragments thereof. Antibodies of the invention include human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind canine CYP1A2 polypeptides exclusively (i.e., able to distinguish canine CYP1A2 polypeptides from other known polypeptides by virtue of measurable differences in binding affinity, despite the possible existence of localized sequence identity, homology, or similarity between canine CYP1A2 and such polypeptides). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the canine CYP1A2 polypeptides of the invention are also contemplated, provided that the antibodies are, first and foremost, specific for canine CYP1A2 polypeptides. Antibodies of the invention can be produced using any method well known and routinely practiced in the art. Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, diagnostic purposes to detect or quantitate canine CYP1A2, as well as purification of canine CYP1A2. Kits comprising an antibody of the invention for any of the purposes described herein are also comprehended. In general, a kit of the invention also includes a control antigen for which the antibody is immunospecific.

### EXAMPLE 3

### Generating Antibodies to Canine CYP1A2

Standard techniques are employed to generate polyclonal or monoclonal antibodies to the canine CYP1A2 enzyme, and to generate useful antigen-binding fragments thereof or variants thereof, including “humanized” variants. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988); and other documents cited below. In one embodiment, recombinant canine CYP1A2 polypeptides (or cells or cell membranes containing such polypeptides) are used as antigen to generate the antibodies. In another embodiment, one or more peptides having amino acid sequences corresponding to an immunogenic portion of canine CYP1A2 (e.g., 6, 7, 8, 9, 10, 11, 12, 13, 14, 15, 16, 17, 18, 19, 20, or more amino acids) are used as antigen In order to mimic a protein epitope with a small synthetic peptide, it is important to choose a sequence that is hydrophilic, surface-oriented, and flexible. This is because most naturally occurring proteins found in physiological solutions have their hydrophilic residues on the surface and their hydrophobic residues buried. Antibodies generally bind to epitopes on the surfaces of naturally occurring proteins. Several known epitopes have a high degree of mobility The N- and C-termini of proteins are generally surface-oriented since they contain charged groups, i.e., NH₃⁺ and COO⁻. They often have a high degree of mobility as well, since they are located at the ends. These termini are often chosen as candidates for synthesis because they possess all three properties. Peptides corresponding to surface residues of canine CYP1A2, especially hydrophilic portions are contemplated. Also contemplated are peptides located at the amino and carboxy terminal ends of canine CYP1A2.

One skilled in the art recognizes that algorithms have been developed to assign values of hydrophilicity, surface accessibility, and flexibility to each amino acid residue within a given protein sequence. The same has been done to assign an antigenic index to each residue, giving an indication of how antigenic that residue is within a specific sequence. Hopp and Woods, Mol. Immunol, 1983 20(4): p. 483-9, Hopp and Woods, Proc. Natl Acad. Sci USA 1981, 78(6) p. 3824-8. Although selection of hydrophilic segments has been widely used in generating anti-peptide antibodies that are useful for binding native antigen. Unlike antibodies however, T cell receptors see relatively small segments of protein antigen after cleavage and unfolding. T cell antigenic sites have also been addressed by predictive computer models. Margalit, H. et al., J. Immunol. 1987 138(7): pg 2213-29.

Computer programs useful for the prediction of epitopes are commercially available. For example MacVector® (Oxford Molecular, Oxford, UK) and Protean® (DNAStar Madison, Wis. 53715). Once a peptide antigen is selected and synthesized the antigen may be mixed with an adjuvant or linked to a hapten to increase antibody production.

Polyclonal or Monoclonal Antibodies

As one exemplary protocol, recombinant canine CYP1A2 or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanine (Pierce), according to the manufacturer's recommendations. For an initial injection, the antigen is emulsified with Freund's Complete Adjuvant and injected subcutaneously. At intervals of two to three weeks, additional aliquots of canine CYP1A2 antigen are emulsified with Freund's Incomplete Adjuvant and injected subcutaneously. Prior to the final booster injection, a serum sample is taken from the immunized mice and assayed by western blot to confirm the presence of antibodies that immunoreact with canine CYP1A2. Serum from the immunized animals may be used as polyclonal antisera or used to isolate polyclonal antibodies that recognize canine CYP1A2. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM Hepes, pH 8.0) (Boehringer Mannheim) is stirred into the pellet, followed by the addition of serum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×106 thymocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusions are screened by ELISA, testing for the presence of mouse IgG that binds to canine CYP1A2. Selected fusion wells are further cloned by dilution until monoclonal cultures producing anti-canine CYP1A2 antibodies are obtained.

Canine CYP1A2 Directed Antibodies from Phage Display

Canine CYP1A2 antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for canine CYP1A2-specific phage-antibodies using labelled or immobilized canine CYP1A2 as antigen-probe.

Canine CYP1A2 Directed Antibodies from Transgenic Mice

Canine CYP1A2 antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with a canine CYP1A2 composition using conventional immunization protocols. Hybridomas are generated using B cells from the immunized mice using conventional protocols and screened to identify hybridomas secreting anti-canine CYP1A2 antibodies (e.g., as described above).

Assays of the Invention

The invention provides methods for detecting or diagnosing the effect of a test agent in a dog in a dog. Dog are treated, preferably at subchronic doses, with one or more known or suspected toxic compounds over a defined time course. Samples of tissue expressing CYP1A2 are then obtained. The level of canine CYP1A2 gene expression derived from such treated biological samples can be compared with the gene expression patterns derived from untreated biological samples to identify compounds which either upregulate or downregulate canine CYP1A2 in response to the compound. The samples can be any sample comprising sample nucleic acid molecules or proteins and obtained from any bodily tissue expressing CYP1A2 (intestine, liver, lung, pancreas, stomach, etc.), cultured cells, biopsies, or other tissue preparations. The level of expression can be assessed at either or both the level of messenger RNA or protein produced.

Nucleic Acid Based Methods

Canine CYP1A2 derived nucleic acids may be in solution or on a solid support. In some embodiments they may be employed as array elements in microarrays alone or in combination with other array element molecules. Such a microarray is particularly useful to detect and characterize gene expression patterns by hybridization associated with test agents known or suspected to be toxic. It is appreciated however, that arrays may be replaced with membrane based hybridization systems or even solution hybridization assays or in fact any method capable of determining specific hybridization. Such gene expression patterns can then be used for comparison to identify other compounds which also elicit a toxicological response. Nucleic acid based methods generally require the isolation of DNA or RNA from the sample and subsequent hybridization or PCR amplification using sprecific primers derived from SEQ ID NO:1. DNA or RNA can be isolated from the sample according to any of a number of methods well known to those of skill in the art. For example, methods of purification of nucleic acids are described in Tijssen, P. (1993) Laboratory Techniques in Biochemistry and Molecular Biology: Hybridization With Nucleic Acid Probes, Part I. Theory and Nucleic Acid Preparation, Elsevier, New York, N.Y. In one preferred embodiment, total RNA is isolated using the TRIZOL total RNA isolation reagent (Life Technologies, Inc., Gaithersburg Md.) and mRNA is isolated using oligo d(T) column chromatography or glass beads. When sample nucleic acid molecules are amplified it is desirable to amplify the sample nucleic acid molecules and maintain the relative abundances of the original sample, including low abundance transcripts. RNA can be amplified in vitro, in situ, or in vivo (See Eberwine U.S. Pat. No. 5,514,545).

It is also advantageous to include controls within the sample to assure that amplification and labeling procedures do not change the true distribution of nucleic acid molecules in a sample. For this purpose, a sample is spiked with an amount of a control nucleic acid molecule predetermined to be detectable upon hybridization to its complementary arrayed nucleic acid molecule and the composition of nucleic acid molecules includes reference nucleic acid molecules which specifically hybridize with the control arrayed nucleic acid molecules. After hybridization and processing, the hybridization signals obtained should reflect accurately the amounts of control arrayed nucleic acid molecules added to the sample.

Prior to hybridization, it may be desirable to fragment the sample nucleic acid molecules. Fragmentation improves hybridization by minimizing secondary structure and cross-hybridization to other sample nucleic acid molecules in the sample or noncomplementary nucleic acid molecules. Fragmentation can be performed by mechanical or chemical means.

Labeling

The sample nucleic acid molecules may be labeled with one or more labeling moieties to allow for detection of hybridized arrayed/sample nucleic acid molecule complexes. The labeling moieties can include compositions that can be detected by spectroscopic, photochemical, biochemical, bioelectronic, immunochemical, electrical, optical or chemical means. The labeling moieties include radioisotopes, such as (32)P, (33)P or (35)S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. Preferred fluorescent markers include Cy3 and Cy5 fluorophores (Amersham Pharmacia Biotech, Piscataway N.J.).

Hybridization

The nulceic acid molecule sequence of SEQ ID NO:1 and fragments thereof can be used in various hybridization technologies for various purposes. Hybridization probes may be designed or derived from SEQ ID NOs:1. Such probes may be made from a highly specific region or from a conserved motif, and used in protocols to quantify CYP1A2 message, allelic variants, or related sequences. The hybridization probes of the subject invention may be DNA or RNA and may be derived from the sequence of SEQ ID NO:1 or from genomic sequences including promoters, enhancers, and introns of the mammalian gene. Hybridization or PCR probes may be produced using oligolabeling, nick translation, end-labeling, or PCR amplification in the presence of the labeled nucleotide. A vector containing the nucleic acid sequence may be used to produce an mRNA probe in vitro by addition of an RNA polymerase and labeled nucleic acid molecules. These procedures may be conducted using commercially available kits such as those provided by Amersham Pharmacia Biotech. The stringency of hybridization is determined by G+C content of the probe, salt concentration, and temperature. In particular, stringency can be increased by reducing the concentration of salt or raising the hybridization temperature. In solutions used for some membrane based hybridizations, additions of an organic solvent such as formamide allows the reaction to occur at a lower temperature. Hybridization can be performed at low stringency with buffers, such as 5×SSC with 1% sodium dodecyl sulfate (SDS) at 60° C., which permits the formation of a hybridization complex between nucleotide sequences that contain some mismatches. Subsequent washes are performed at higher stringency with buffers such as 0.2×SSC with 0.1% SDS at either 45° C. (medium stringency) or 68° C. (high stringency). At high stringency, hybridization complexes will remain stable only where the nucleic acid sequences are almost completely complementary. In some membrane-based hybridizations, preferably 35% or most preferably 50%, formamide can be added to the hybridization solution to reduce the temperature at which hybridization is performed, and background signals can be reduced by the use of other detergents such as Sarkosyl or Triton X-100 and a blocking agent such as salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Exemplary highly stringent hybridization conditions are as follows: hybridization at 42° C. in a hybridization solution comprising 50% formamide, 1% SDS, 1M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

Hybridization specificity can be evaluated by comparing the hybridization of specificity-control nucleic acid molecules to specificity-control sample nucleic acid molecules that are added to a sample in a known amount. The specificity-control arrayed nucleic acid molecules may have one or more sequence mismatches compared with the corresponding arrayed nucleic acid molecules. In this manner, it is possible to determine whether only complementary arrayed nucleic acid molecules are hybridizing to the sample nucleic acid molecules or whether mismatched hybrid duplexes are forming is determined.

Hybridization reactions can be performed in absolute or differential hybridization formats. In the absolute hybridization format, nucleic acid molecules from one sample are hybridized to the molecules in a microarray format and signals detected after hybridization complex formation correlate to nucleic acid molecule levels in a sample. In the differential hybridization format, the differential expression of a set of genes in two biological samples is analyzed. For differential hybridization, nucleic acid molecules from both biological samples are prepared and labeled with different labeling moieties. A mixture of the two labeled nucleic acid molecules is added to a microarray. The microarray is then examined under conditions in which the emissions from the two different labels are individually detectable. Molecules in the microarray that are hybridized to substantially equal numbers of nucleic acid molecules derived from both biological samples give a distinct combined fluorescence (Shalon et al. PCT publication WO95/35505). In a preferred embodiment, the labels are fluorescent markers with distinguishable emission spectra, such as Cy3 and Cy5 fluorophores.

After hybridization, the microarray is washed to remove nonhybridized nucleic acid molecules and complex formation between the hybridizable array elements and the nucleic acid molecules is detected. Methods for detecting complex formation are well known to those skilled in the art. In a preferred embodiment, the nucleic acid molecules are labeled with a fluorescent label and measurement of levels and patterns of fluorescence indicative of complex formation is accomplished by fluorescence microscopy, preferably confocal fluorescence microscopy.

In a differential hybridization experiment, nucleic acid molecules from two or more different biological samples are labeled with two or more different fluorescent labels with different emission wavelengths. Fluorescent signals are detected separately with different photomultipliers set to detect specific wavelengths. The relative abundances/expression levels of the nucleic acid molecules in two or more samples is obtained.

Typically, microarray fluorescence intensities can be normalized to take into account variations in hybridization intensities when more than one microarray is used under similar test conditions. In a preferred embodiment, individual arrayed-sample nucleic acid molecule complex hybridization intensities are normalized using the intensities derived from internal normalization controls contained on each microarray.

Polypeptide Based Assays

The present invention provides methods and reagents for detecting and quantifying canine CYP1A2 polypeptides. These methods include analytical biochemical methods such as electrophoresis, mass spectroscopy, chromatographic methods and the like, or various immunological methods such as radioimmunoassay (RIA), enzyme-linked immunosorbent assays (ELISAs), immunofluorescent assays, western blotting, affinity capture mass spectrometry, biological activity and others described below and apparent to those of skill in the art upon review of this disclosure.

Immunoassays

The present invention also provides methods for detection of canine CYP1A2 polypeptides employing one or more anti-canine CYP1A2 antibody reagents (i.e., immunoassays). As used herein, an immunoassay is an assay that utilizes an antibody (as broadly defined herein and specifically includes fragments, chimeras and other binding agents) that specifically binds a canine CYP1A2 polypeptide or epitope.

A number of well established immunological binding assay formats suitable for the practice of the invention are known (see, e.g., U.S. Pat. Nos. 4,366,241; 4,376,110; 4,517,288; and 4,837,168). See, e.g., Methods in Cell Biology Volume 37: Antibodies in Cell Biology, Asai, ed. Academic Press, Inc. New York (1993); Basic and Clinical Immunology 7th Edition, Stites & Terr, eds. (1991); Harlow and Lane, supra [e.g., Chapter 14], and Ausubel et al., supra, [e.g., Chapter 11]. Typically, immunological binding assays (or immunoassays) utilize a “capture agent” to specifically bind to and, often, immobilize the analyte to a solid phase. In one embodiment, the capture agent is a moiety that specifically binds to a canine CYP1A2 polypeptide or subsequence, such as an anti-canine CYP1A2 antibody. Usually the canine CYP1A2 gene product being assayed is detected directly or indirectly using a detectable label. The particular label or detectable group used in the assay is usually not a critical aspect of the invention, so long as it does not significantly interfere with the specific binding of the antibody or antibodies used in the assay. The label may be covalently attached to the capture agent (e.g., an anti-canine CYP1A2 antibody), or may be attached to a third moiety, such as another antibody, that specifically binds to the canine CYP1A2 polypeptide.

The present invention provides methods and reagents for competitive and noncompetitive immunoassays for detecting canine CYP1A2 polypeptides. Noncompetitive immunoassays are assays in which the amount of captured analyte (in this case canine CYP1A2) is directly measured. One such assay is a two-site, monoclonal-based immunoassay utilizing monoclonal antibodies reactive to two non-interfering epitopes on the canine CYP1A2 protein. See, e.g., Maddox et al., 1983, J. Exp. Med., 158:1211 for background information. In one “sandwich” assay, the capture agent (e.g., an anti-canine CYP1A2 antibody) is bound directly to a solid substrate where it is immobilized. These immobilized antibodies then capture any canine CYP1A2 protein present in the test sample. The canine CYP1A2 thus immobilized can then be labeled, i.e., by binding to a second canine-CYP1A2 antibody bearing a label. Alternatively, the second canine CYP1A2 antibody may lack a label, but be bound by a labeled third antibody specific to antibodies of the species from which the second antibody is derived. The second antibody alternatively can be modified with a detectable moiety, such as biotin, to which a third labeled molecule can specifically bind, such as enzyme-labeled streptavidin.

In competitive assays, the amount of canine CYP1A2 protein present in the sample is measured indirectly by measuring the amount of an added (exogenous) canine CYP1A2 displaced (or competed away) from a capture agent (e.g., canine CYP1A2 antibody) by the canine CYP1A2 protein present in the sample. A hapten inhibition assay is another example of a competitive assay. In this assay canine CYP1A2 protein is immobilized on a solid substrate. A known amount of canine CYP1A2 antibody is added to the sample, and the sample is then contacted with the immobilized canine CYP1A2 protein. In this case, the amount of anti-canine CYP1A2 antibody bound to the immobilized canine CYP1A2 protein is inversely proportional to the amount of canine CYP1A2 protein present in the sample. The amount of immobilized antibody may be detected by detecting either the immobilized fraction of antibody or the fraction of the antibody that remains in solution. In this aspect, detection may be direct, where the antibody is labeled, or indirect where the label is bound to a molecule that specifically binds to the antibody as described above.

Other Antibody-Based Assay Formats

The invention also provides reagents and methods for detecting and quantifying the presence of canine CYP1A2 polypeptide in the sample by using an immunoblot (Western blot) format. Another immunoassay is the so-called “lateral flow chromatography.” In a non-competitive version of lateral flow chromatography, a sample moves across a substrate by, e.g., capillary action, and encounters a mobile labeled antibody that binds the analyte forming a conjugate. The conjugate then moves across the substrate and encounters an immobilized second antibody that binds the analyte. Thus, immobilized analyte is detected by detecting the labeled antibody. In a competitive version of lateral flow chromatography a labeled version of the analyte moves across the carrier and competes with unlabeled analyte for binding with the immobilized antibody. The greater the amount of the analyte in the sample, the less the binding by labeled analyte and, therefore, the weaker the signal. See, e.g., May et al., U.S. Pat. No. 5,622,871 and Rosenstein, U.S. Pat. No. 5,591,645.

Depending upon the assay, various components, including the antigen, target antibody, or anti-cathepsin S antibody, may be bound to a solid surface or support (i.e., a substrate, membrane, or filter paper). Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g. glass, PVC, polypropylene, polystyrene, latex, and the like), a microcentrifuge tube, or a glass or plastic bead. The desired component may be covalently bound or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which may be employed, include paper, glasses, ceramics, metals, metalloids, semiconductive materials, cements or the like. In addition, substances that form gels, such as proteins (e. g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

Mass Spectrometry

The mass of a molecule frequently can be used as an identifier of the molecule. Therefore, methods of mass spectrometry can be used to identify a protein analyte. Mass spectrometers can measure mass by determining the time required for an ionized analyte to travel down a flight tube and to be detected by an ion detector. One method of mass spectrometry for proteins is matrix-assisted laser desorption ionization mass spectrometry (“MALDI”). In MALDI the analyte is mixed with an energy absorbing matrix material that absorbs energy of the wavelength of a laser and placed on the surface of a probe. Upon striking the matrix with the laser, the analyte is desorbed from the probe surface, ionized, and detected by the ion detector. See, for example, Hillenkamp et al., U.S. Pat. No. 5,118,937.

Other methods of mass spectrometry for proteins are described in Hutchens and Yip, U.S. Pat. No. 5,719,060. In one such method referred to as Surfaces Enhanced for Affinity Capture (“SEAC”) a solid phase affinity reagent that binds the analyte specifically or non-specifically, such as an antibody or a metal ion, is used to separate the analyte from other materials in a sample. Then the captured analyte is desorbed from the solid phase by, e.g., laser energy, ionized, and detected by the detector.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

I claim:

1. An isolated canine CYP1A2 polypeptide wherein the amino acid sequence comprises SEQ ID NO:2, or a fragment thereof comprising an epitope specific to said polypeptide.

2. An antibody specific for the canine CYP1A2 polypeptide of claim 1.

3. The antibody of claim 2 which is a monoclonal antibody.

4. A hybridoma that produces an antibody according to claim 3.

5. A cell-free composition comprising monoclonal antibodies, wherein at least one of said antibodies is an antibody according to claim 3.

6. An isolated polynucleotide comprising SEQ ID NO: 1 or a fragment thereof comprising at least 12 consecutive nucleotides of SEQ ID NO:1 or the non-coding strand complementary thereto with the provision that said fragment comprises a nucleotide sequence that differs from any portion of the sequences set forth as SEQ ID NO: 9, 11, 13 and from their complementary strands by at least one nucleotide.

7. The isolated nucleic acid of claim 6 wherein the nucleic acid is single stranded.

8. The isolated nucleic acid of claim 6 comprising at least 12 consecutive nucleotides of the complement of SEQ ID NO:1.

9. An array of nucleic acid molecules, attached to a solid support, the array comprising the polynucleotide of claim 6.

10. An isolated polynucleotide comprising a nucleotide sequence that encodes a polypeptide comprising SEQ ID NO:2.

11. The isolated polynucleotide of claim 10 which is SEQ ID NO:1.

12. A method for determining the amount of canine CYP1A2 polynucleotide present within a sample derived from a dog comprising:
contacting the sample with a nucleic acid molecule comprising SEQ ID NO:1 or fragments thereof or their complements under conditions for the formation of one or more specific hybridization complexes, wherein the fragments are polynucleotides comprising at least 12 consecutive nucleotides of SEQ ID NO:1

13. A method for measuring the metabolic response to a test agent in a dog comprising:
a) providing a sample containing nucleic acids from a dog treated with a test agent;
b) determining the amount of polynucleotide comprising SEQ ID NO:1, or a fragment thereof or their complements in said sample, wherein a change in the amount of the polynucleotide from a treated dog, as compared with the amount of the polynucleotide from an untreated dog, is indicative of a metabolic response to the test agent.

14. The method of claim 13 wherein the determining is accomplished via hybridization.

15. The method of claim 14 wherein the hybridization is accomplished by:
a) contacting the sample with a nucleic acid molecule comprising SEQ ID NO:1 or fragments or their complements thereof under conditions for the formation of one or more specific hybridization complexes, wherein the fragments are polynucleotides comprising at least 12 consecutive nucleotides of SEQ ID NO:1; and
b) detecting hybridization complexes, wherein a change in amount of hybridization complexes formed from nucleic acid molecules from an treated dog, as compared with the amount of hybridization complexes formed from nucleic acid molecules from an untreated dog, is indicative of a metabolic response to the test agent.

16. The method of claim 15 wherein the nucleic acid molecule is attached to a solid support.

17. A method for determining the amount of canine CYP1A2 polypeptide present within a sample comprising:
contacting a canine CYP1A2 polypeptide with an antibody specific for the canine CYP1A2 polypeptide, under conditions wherein the antibody binds the canine CYP1A2 polypeptide.

18. The method of claim 17 wherein the canine CYP1A2 polypeptide is attached to a solid support.

19. The method of claim 17 wherein the antibody is attached to a solid support.

20. A method for measuring the metabolic response to a test agent in a dog comprising:
a) providing a sample from a dog treated with a test agent;
b) determining the amount of polypeptide comprising SEQ ID NO:2, or a fragment thereof comprising an epitope specific to said polypeptide in said sample, wherein a change in the amount of the polypeptide from a treated dog, as compared with the amount of the polypeptide from an untreated dog, is indicative of a metabolic response to the test agent.

21. The method of claim 20 wherein the determining is accomplished by contacting a canine CYP1A2 polypeptide with an antibody specific for the canine CYP1A2 polypeptide, under conditions wherein the antibody binds the canine CYP1A2 polypeptide.
